SEQUENCE LISTING (4 sequences)

SEQ ID NO: 1 - DNA, 25 bp, Homo sapiens
  RT-PCR primer of GAPDH
tgatgacatcaagaaggtggtgaag

SEQ ID NO: 2 - DNA, 23 bp, Homo sapiens
  RT-PCR primer of GAPDH
tccttggaggccatgtgggccat

SEQ ID NO: 3 - DNA, 21 bp, Homo sapiens
  RT-PCR primer of GST-1
tcactaaagcctcctgcctat

SEQ ID NO: 4 - DNA, 18 bp, Homo sapiens
  RT-PCR primer of GST-1
gccttcacatagtcatcc

(19) United States
(12) Patent Application Publication
(10) Pub. No.: US 2013/0156701 A1
(43) Pub. Date: Jun. 20, 2013

(54) METHOD OF PREPARING ETHACRYNIC AMIDE DERIVATIVES AND APPLICATION THEREOF

(71) Applicant: National Tsing Hua University, Hsinchu (TW)

(72) Inventors: CHUNG SHAN YU, Hsinchu (TW); CHUN NAN YEH, Hsinchu (TW); JENN-TZONG CHEN, Hsinchu (TW); WUU-JYH LIN, Hsinchu (TW); YIN-CHENG HUANG, Hsinchu (TW); LI-WU CHIANG, Hsinchu (TW); HAO LIEN HUANG, Hsinchu (TW); KANG-WEI CHANG, Hsinchu (TW); GON-SHEN CHEN, Hsinchu (TW); KEN-HONG LIN, Hsinchu (TW); WEI-TING WANG, Hsinchu (TW)

(73) Assignee: NATIONAL TSING HUA UNIVERSITY, Hsinchu (TW)

(21) Appl. No.: 13/715,513

(22) Filed: Dec. 14, 2012

(30) Foreign Application Priority Data
Dec. 15, 2011 (TW) 100146538
Jun. 28, 2012 (TW) 101123252

(51) Int. Cl. C07C 231/12; A61K 51/04

(52) U.S. Cl. CPC C07C 231/12; A61K 51/04
USPC 424/1.89; 564/169

(57) ABSTRACT

The present invention provides a method for preparing [¹⁸F]—N-(4-fluorobutyl)ethacrynic amide which is prepared from radiofluorination and deprotection of the precursor tosylate N-Boc-N-[4-(toluenesulfonyloxy)-butyl)ethacrynic amide], obtained from ethacrynic acid via 6-step synthesis in 39% yield, in a radiochemical yield of 44%, aspecific activity of 48 GBq/μmol and radiochemical purity of 98%. The present invention further provides a composition for positron emission tomography (PET) of an animal models of a tumor liver or a liver disease, comprising [¹⁸F]—N-(4-fluorobutyl)ethacrynic amide and a pharmaceutically acceptable carrier.

### CROSS-REFERENCES TO RELATED APPLICATIONS

This application claims priority to Taiwan Patent Application No. 100146538 filed on 15 Dec. 2011 and Taiwan Patent Application No. 101123252 filed on 28 Jun. 2012. All disclosure of which is incorporated herein by reference in its entirety.

### BACKGROUND OF THE INVENTION

1. Field of the Invention

The present invention relates to a method of preparing ethacrynic amine derivatives and application thereof.

2. The Prior Arts

Presently, positron emission tomography (PET) is a medicine imaging technique that produced a three-dimensional image or picture of functional processes in the body. PET is applied heavily in medical image of tumors and the search for metastases, and molecules (e.g. drugs) and biological macromolecules (e.g. proteins) in vivo imaging rely on the positron emitters (e.g. fluoro-18). The characteristics of ¹⁸F, such as low radiation doses, short tissue range, feasibility of multi-step synthesis and extendable scanning protocols are attributed to a relatively low energy of 0.64 MeV and a relatively long half-life (t_{1/2}=109.7 min). The adequate atomic size due to being a member of the second periodic atoms makes ¹⁸F a suitable atom for mimicking oxygen or hydrogen. The high sensitivity of ¹⁸F allows the use of a very low concentration (10⁻¹² M) of radio-labeled tracer for imaging cellular markers, for example, receptors without encountering toxicity concerns.

Introduction of ¹⁸F can be mediated through a direct substitution reaction or an indirect reaction via a bifunctional group. The former includes a nucleophilc or electrophilic pathway. The bifunctional group is also named prosthetic group or synthon of ¹⁸F. Previously research has indicated that a substituted butyl ethacrynic acid (EA) analog which shows a modified cytotoxicity is different from EA (FIG. 1, Chiang et al. (2009) Chem. Pharm. Bullet. 57, 714-718).

EA with the conjugated ketene can act as an electron sink attacked by thiol in Michael addition reaction, GSH-EA complex of EA and glutathione (GSH) has more inhibition to glutathione S-transferase (GST) than EA (Wortelboer et al. (2003) Chem. Res. Toxicol. 16, 1642-1651, Ploemen et al. (1990) Biochem. Pharmacol. 40, 1631-1635). Presently, GSTs are encode by seven distantly related gene family within types of cells in vertebrates (designated class: α, π, μ, θ, ω, ζ and σ).

As an important antioxidant, GSH plays a role in the detoxification of a variety of electrophilic compounds and peroxides via catalysis by GST family of enzymes and glutathione peroxidases (GPx). In different tissue, the expression level of GST is also different, for example, GST-α expresses at high level in liver, testis and kidney; and GST-π expresses at high level in brain, lung, heart, or even in cancer cells. GST family's potential is as a compelling drug target due to the cytoprotective effect and resistance to the anticancer agents (Laborde (2010) Cell Death Differ 17, 1373-1380).

EA can effectively increase the cells in vitro (in cell cultures) or even in vivo (in patient tissues) sensitivity to melphalan, piriprost or chlorambucil. But its potential toxicity and diuretic effects limit the application of EA in medical. However, enol of EA undergoes nucleophilic attack by thiol of GSH, EA still can be a ¹⁸F radio-labeled tracer in the vivo for imaging GST activity.

Taiwan Patent Application No. 098218614 has disclosed N-(4-[¹⁸F]-fluorobutyl)ethacrynic amide ([¹⁸F]FBuEA) as well as the method for preparing its precursor and non-radioactive standard prepared and charactered by HPLC. However, the patent only discloses the method for preparing the precursor of [¹⁸F]FBuEA, it discloses neither the method for preparing [¹⁸F]FBuEA nor the applying [¹⁸F]FBuEA in nuclear medicine imaging. Thus, it obviously needs a method for preparing [¹⁸F]FBuEA and further application.

### SUMMARY OF THE INVENTION

As the discovery of BuEA, it promotes to develop an in situ BuEA-based screening of the library (Su et al. (2011) Bioorg. Med. Chem. Lett. 21, 1320-1324). Using the derivative of ethacrynic acid butyl amide members in the library more than 100 compounds analyzes the cytotoxicities to tumor cells (e.g. A549, MCF-7, TRAMP-C1 and C26). No compound is found to have a good bioactivity substance. While the structure of EA-butyl ester analogs is similar to BuEA, the lipophilic butyl group may increase the ability of the passive penetrating cells. The selective cytotoxicity of EA-butyl ester analogs is considered to be the most likely target. Therefore, the present invention is to prepare ¹⁸F-labeled BuEA analogs as a substrate to assess its potential use as an in vivo as an imaging agent.

The structure of FBuEA shows that fluorine at the end position of the butyl group should not alter the enone functionality. In addition, the precursor of the radiofluorination is primary alcohol-derived tosylate which radiates fluoride via the S_{N}2 mechanism. Therefore, FBuEA has been widely used for radiofluorination in radochemstry.

Preparation of compounds may involve the protection or deprotection in various chemical groups. The protection and deprotection are required to select the suitable protection group which can be determined by those skilled in the art.

The invention provides a method for preparing the compound of formula 1, [¹⁸F]fluorobutyl ethacrynic amid ([¹⁸F]FBuEA)

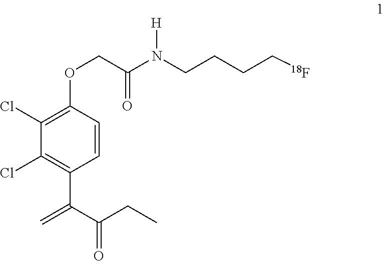 [SMILES: C=C(C(=O)CC)c1ccc(OCC(=O)NCCCC[18F])c(Cl)c1Cl]

the method comprises: (a) reacting the compound of formula 2

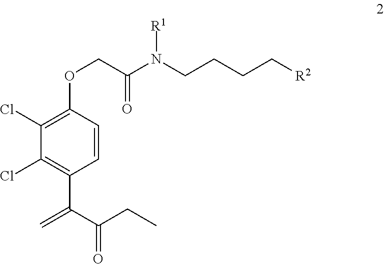

with ¹⁸F-labeled fluorine reagent and acetonitrile to form the compound of formula 3; and

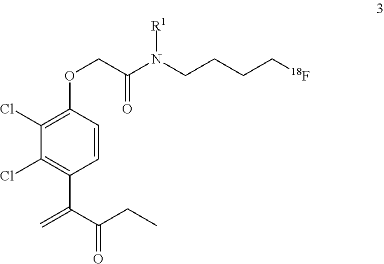

(b) using the compound of formula 3 with trifluoro acetic acid and haloalkanes to form the compound of formula 1; wherein R¹ is a protecting group of amide functional group and R² is a leaving group; the protecting group of amide functional group preferably is a tert-butoxycarbonyl; the leaving group preferably is tosyloxy, methanesulfonyl, trifluoromethanesulfonyloxyl or bromine; ¹⁸F-labeled fluorine reagent preferably is ¹⁸F-labeled tetrabutyl ammonium fluoride.

The compound of formula 2 is formed by reacting the compound of formula 4

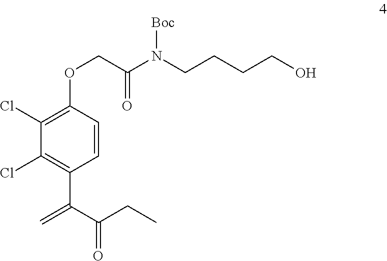 [SMILES: C=C(C(=O)CC)c1ccc(OCC(=O)N(CCCCO)C(=O)OC(C)(C)C)c(Cl)c1Cl]

with toluenesulfonyl chloride and a pyridine compound; Boc is tert-butoxycarbonyl; the pyridine compound preferably is 4-(dimethylamino)pyridine.

The compound of formula 4 is formed by the compound of formula 5

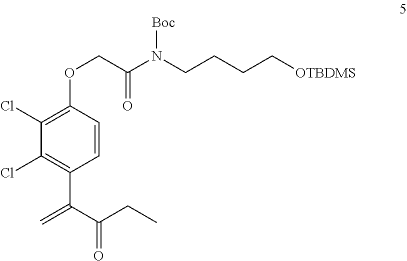 [SMILES: C=C(C(=O)CC)c1ccc(OCC(=O)N(CCCCO[Si](C)(C)C(C)(C)C)C(=O)OC(C)(C)C)c(Cl)c1Cl]

with tetrabutyl ammonium fluoride and acetic acid; Boc is a tert-butoxycarbonyl; OTBDMS is tert-butdimethoxysilane.

The compound of formula 5 is formed by reacting the compound of formula 6

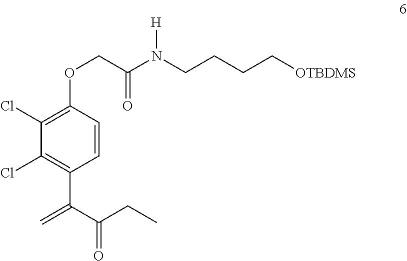 [SMILES: C=C(C(=O)CC)c1ccc(OCC(=O)NCCCCO[Si](C)(C)C(C)(C)C)c(Cl)c1Cl]

and di-tert-butyl dicarbonate; OTBDMS is tert-butdimethoxysilane.

The compound of formula 6 is formed by reacting ethacrynic acid with N-Boc-N-[4-(t-butyldimethylsilanyloxy)butyl-1-amine.

The present invention also provides a composition for positron emission tomography (PET) imaging, the composition comprises the compound of formula 1 and a pharmaceutically acceptable carrier, wherein the positron emission tomography (PET) imaging is used in an animal model of a liver tumor or a liver disease, and the liver disease preferably is cirrhosis.

The present invention provides a method for PET imaging in liver, comprising: (a) prepare to scan a subject with PET system; (b) inject above-mentioned composition into the subject; (c) image the liver of the subject and confirm the cold spot of the non-radiated signal in the image. The method can be used in model of animal liver tumor or liver disease, wherein the model of liver disease may preferably becirrhosis liver.

Moreover, the present invention relates to processes for preparing a precursor of [¹⁸F]FBuEA. It is simpler than prior-art processes. Overall, the invention can reduce more time and cost than the preparation of prior-art processes.

The detailed technology and above preferred embodiments implemented for the present invention are described in the following paragraphs accompanying the appended drawings for people skilled in this field to well appreciate the features of the claimed invention.

### BRIEF DESCRIPTION OF THE DRAWINGS

FIG. 1 shows the cytotoxicities of ethacrynic acid analogs versus A549 cells.

FIG. 2(A) and (B) are chromatograms for [¹⁸F]FBuEA before (A) and after (B) HPLC purification. A normal phase semipreparative column (Si-100) was employed, AU: Arbitrary Unit, CPS: count per second.

FIG. 3 is RP-HPLC chromatogram for the mixtures of [¹⁸F]FBuEA-GSH (t_{R}=17.5 min) and the residual [¹⁸F]FBuEA (t_{R}=23.7 min) obtained from self conjugation.

FIG. 4 is RP-HPLC chromatogram for the mixtures of [¹⁸F]FBuEA-GSH (t_{R}=16.5 min) by coinjection with non-radiated authentic FBuEA-GSH under catalysis of GST-π. The peak at t_{R}=6.5 min is suggested to be GST-π;

FIG. 5(A) shows that trypan blue exclusion assay is performed to quantity the cell viability with different concentration of FBuEA for 48 hr, the IC₅₀ of 293T, A549 and HEL cells is 20 μM, 14 μM and 5 μM, respectively.

FIG. 5(B) shows RT-PCR transcriptome analysis of GST-π1 and GAPDH in 293T, A549 and HEL cells.

FIG. 5(C) shows expression levels of GST-π1 (RT-PCR) in 293T, A549 and HEL cells are normalized by expression level of GAPDH.

FIGS. 6 (A) to (C) show metabolite analysis of [¹⁸F]FBuEA, HPLC chromatogram of serum samples from heart are taken at each time point 10 min, 30 min and 60 min after [¹⁸F]FBuEA injection, and there is no radioactivity in serum sample at 90 min;

FIG. 6 (D) shows [¹⁸F]FBuEA time-activity relationship obtained by integrating the counts of the peak corresponding to [¹⁸F]FBuEA from the chromatogram taken at each time point. Plasma T_{1/2}=46 min. CPS: counts pre second.

FIG. 7 shows micro PET images of the rat using [¹⁸F]FBuEA at different time frame.

FIG. 8 is an image of radioactivity accumulation in the region of interest (ROI) for calculating time-radioactivity relationship.

FIG. 9 is a curve of time-radioactivity relationship of PET images in brain, tumor, liver, kidney and bladder post injection.

FIG. 10(A) shows PET images of the normal rat are normalized from 0 to 120 post injection.

FIG. 10(B) shows PET images of the CCA rat are normalized from 0 to 120 post injection.

FIG. 11(A) shows PET imaging of the normal rat using [¹⁸F]FBuEA are average of dynamic images of time frame from 0 to 30 min.

FIG. 11(B) shows PET images of the CCA rat using [¹⁸F]FBuEA are average of dynamic images of time frame from 0 to 30 min.

FIG. 11(C) PET images of the same CCA rat as that of (B) are taken by using [¹⁸F]FDG post 90 min. Arrow indicated the lesion of tumor.

FIG. 11(D) PET images of the normal rat are average of dynamic image of time frame from 5 to 10 min post injection.

FIG. 11(E) PET images of the same rat as that described in (B) with a prolonged feeding to 23 weeks of TAA are average of dynamic images of time frame from 5 to 10 min post injection.

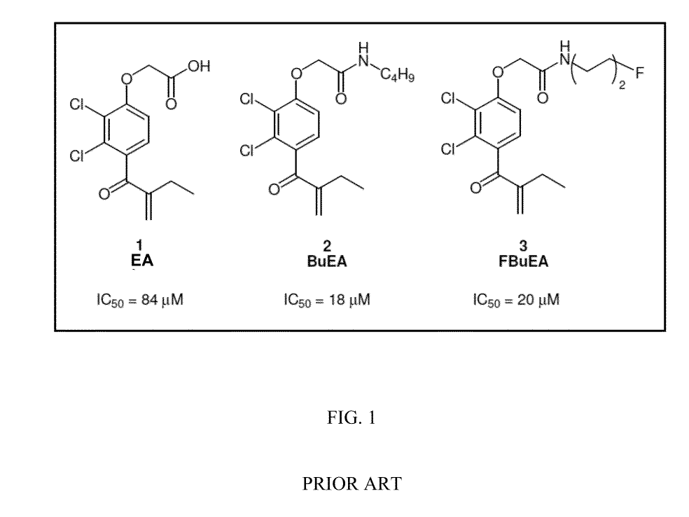

### DETAILED DESCRIPTION OF THE INVENTION

### Example 1

### 1. Chemical Synthesis

All reagents and solvents were purchased from Sigma-Aldrich, Malingkrodt, Acros, Alfa, Tedia, or Fluka. All preparations for non-radioactive compounds were routinely conducted in dried glassware under a positive pressure of nitrogen at room temperature unless otherwise noted. CH₂Cl₂, toluene, CH₃CN, and pyridine were dried over CaH₂ and MeOH was dried over Mg and distilled prior to reaction. DMF and NEt₃ were distilled under reduced pressure. Reagents and solvents were of reagent grade. Dimethyl amino pyridine (DMAP) was purified through recrystallization from the combination of EtOAc and n-hexane before use. The eluents for chromatography: EtOAc, acetone, and n-hexane were reagent grade and distilled prior to use; MeOH and CHCl₃ were reagent grade and used without further purification. NMR spectroscopy including ¹H-NMR (500 MHz) and ¹³C-NMR (125 MHz, DEPT-135) was measured on Varian UnityInova 500 MHz. D-solvents employed for NMR including CD₃OD, CDCl₃, C₆D₆, and DMSO-d⁶ were purchased from Cambridge Isotope Laboratories, Inc. Low-resolution mass spectrometry (LRMS) was performed on a ESI-MS spectrometry employing VARIAN 901-MS Liquid Chromatography Tandem Mass Q-T of Spectrometer was performed at the department of chemistry of National Tsing-Hua University (NTHU). High-resolution mass spectrometry (HRMS) was performed using a varian HPLC (prostar series ESI/APCI) coupled mass detector of Varian 901-MS (FT-ICR Mass) and triple quadrapole. Thin layer chromatography (TLC) was performed with MERCK TLC Silica gel 60 F₂₅₄ precoated plates. The starting compounds and products were visualized with UV light (254 nm). Further confirmation was carried out by using staining with 5% p-anisaldehyde, ninhydrin or ceric ammonium molybdate under heating. Flash chromatography was performed using Geduran Si 60 silica gel (230-400 mesh). Melting points were measured with MEL-TEMP and were uncorrected.

### 2. 4-(tert-butyldimethylsilanyloxy)butan-1-amine (compound of formula 7)

Preparation of this compound was according to the procedure reported by Krivickas S. J. et al. Tert-Butyldimethylsiyl Chloride (TBDMSCl) (8.2 g, 54 mmol, 1.2 eq) was added to a mixture of 4-aminobutanol (4 g, 45 mmol) and pyridine (8 mL). Stirring was allowed for 12 h. TLC (MeOH/CHCl₃=5/5) indicated the consumption of 4-aminobutanol (R_{f}=0.13) and formation of compound of formula 7 (R_{f}=0.40). The mixture was then concentrated under reduced pressure at 40° C. to provide a residue, which was dissolved by CH₂Cl₂ (50 mL). After extraction with satd. NaHCO₃ (aq), the organic layer was dried over Na₂SO₄ and filtered through celite pad to provide the filtrate, which was concentrated under reduced pressure. The residue obtained was purified by flash chromatography using silica gel (50 g) with eluents of Et₃N/MeOH/CHCl₃=2/10/90 to provide a colorless oil compound of formula 7 in quantitative yield (8.8 g). Spectroscopic data is available in the literature. Anal. C₁₀H₂₅NOSi, MW: 203.4, ESI+Q-TOF MS, M=203.2 (m/z), [M+H]⁺=204.2; ¹H-NMR (500 MHz, CD₃OD): δ 0.06 (s, 6H, H_{TBDMS}), 0.90 (s, 9H, H_{TBDMS}), 1.55-1.59 (m, 4H), 2.74 (dd, 2H), 3.66 (dd, 2H).

### 3. N-[4-(t-butyldimethylsilanyloxy)butyl]ethacrynic amide (compound of formula 6)

Ethacrynic acid (EA) (1.2 g, 4 mmol) was dried through azeotropical distillation with toluene three times. After mixing with DMF (2 mL), the mixture was transferred to a two-neck round-bottom flask followed by charging with O-Benzotriazol-1-yl-tetramethyluronium (HBTU) (1.65 g, 4.4 mmol, 1.1 eq), N,N-Diisopropylethylamine (DIEA) (0.76 mL, 4.4 mmol, 1.1 eq) and compound of formula 7 (805 mg, 4 mmol, 1 eq) sequentially. Stirring was allowed for 15 min. TLC (MeOH/CHCl₃=2/8) indicated the consumption of EA (R_{f}=0.13) and formation of the compound of formula 6 (R_{f}=0.40). The mixture was then concentrated at 40° C. under reduced pressure and the residue obtained was purified further by flash chromatography using silica gel (100 g) with eluents of EtOAc/n-hexane 3/7 to provide a colorless oil compound of formula 6 in 70% yield (1.35 g). Anal. C₂₃H₃₅Cl₂NO₄Si, MW: 488.5, ESI+Q-TOF MS, M=487.2 (m/z), [M+H]⁺=488.2, [M+Na]⁺=510.1, [2M+Na]⁺=997.4; the isotope clusters agree with the presence of Cl×2. ¹H-NMR (500 MHz, C₆D₆): δ 0.04 (s, 6H, H_{TBDMS}), 0.96 (s, 9H, H_{TBDMS}), 1.02 (dd, J=7.5 Hz, 3H, CH₂CH₃), 1.36-1.48 (m, 4H, CH₂), 2.42 (q, J=7.5 Hz, 2H, CCH₂CH₃), 3.18 (q, J=6.5 Hz, 2H, (CONH)CH₂CH₂), 3.45 (t, J=6.0 Hz, 2H, CH₂CH₂OTBDMS), 3.91 (d, J=5.0 Hz, 2H, O(CH₂)CONH), 5.26 (s, 1H, C═CH₂), 5.43 (s, 1H, C═CH₂), 5.85-5.90 (m, 1H, Hₐᵣₒₘ), 6.36 (bs, 1H, NH), 6.63 (dd, J=8.5, 2.0 Hz, 1H, Hₐᵣₒₘ). ¹³C-NMR (125 MHz, C₆D₆): δ −5.24 (CH₃, TBDMS), 12.63 (CH₂CH₃), 18.44 (C, TBDMS), 23.90 (CH₂CH₃), 26.10 (CH₃, TBDMS), 26.60 (CH₂), 30.16 (CH₂), 38.89 (CH₂), 62.70 (CH₂), 68.50 (CH₂), 111.13 (CH, arom), 122.83 (C, C═CH₂), 127.29 (CH, arom), 127.57 (CH₂, C═CH₂), 131.42 (C, arom), 134.42 (C, arom), 150.64 (C, arom), 154.72 (C, arom), 165.87 (C, C═O), 194.76 (C, C═O).

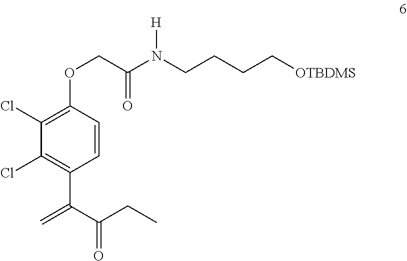 [SMILES: C=C(C(=O)CC)c1ccc(OCC(=O)NCCCCO[Si](C)(C)C(C)(C)C)c(Cl)c1Cl]

### 4. N-Boc-N-[4-(t-butyldimethylsilanyloxy)butyl]ethacrynic amide (compound of formula 5)

The starting compound of formula 6 (682 mg, 1.40 mmol) was dried through azeotropical distillation with toluene at 40° C. three times. After mixing with CH₃CN (10 mL), the mixture was transferred to a two-neck round-bottom flask followed by charging with Boc₂O (0.64 mL, 2.80 mmol, 2 eq), Et₃N (0.27 mL, 1.96 mmol, 1.4 eq), and dimethyl aminopyridine (273 mg, 2.24 mmol, 1.6 eq) sequentially. Stirring was allowed for 6 h. TLC (EtOAc/n-hexane 3/7) indicated the consumption of compound of formula 6 (R_{f}=0.40) and formation of the compound of formula 5 (R_{f}=0.73). The mixture was then concentrated at 40° C. under reduced pressure followed by purification by flash chromatography using silica gel (80 g) with eluents of EtOAc/n-hexane 1/9 to provide a colorless oil compound of formula 5 in 76% yield (626 mg). Anal. C₂₈H₄₃Cl₂NO₆Si, MW: 588.6, ESI+Q-TOF MS, M=587.2 (m/z), [M-Boc+H]⁺=488.2, [M+H]⁺=588.2, [M+Na]⁺=610.3; the isotope clusters agree with the presence of Cl×2. HRMS-ESI, Calcd. C₂₈H₄₃Cl₂NO₆Si [M]: 587.22367. found: 587.21601. ¹H-NMR (500 MHz, C₆D₆): δ 0.00 (s, 6H, H_{TBDMS}), 0.95 (s, 9H, H_{TBDMS}), 0.98 (t, J=7.5 Hz, 3H, CH₂CH₃), 1.30 (s, 9H, H_{Boc}), 1.38-1.42 (m, CH₂CH₂OTBDMS), 1.59-1.63 (m, (CON)CH₂CH₂), 2.43 (ddd, J=7.5 Hz, 2H, CCH₂CH₃), 3.45 (t, J=7.0 Hz, 2H, (CON)CH₂CH₂), 3.62 (t, J=6.0 Hz, 2H, CH₂CH₂OTBDMS), 5.00 (s, 2H, O(CH₂)CON), 5.25 (s, 1H, C═CH₂), 5.42 (s, 1H, C═CH₂), 6.24 (d, J=8.5 Hz, 1H, Hₐᵣₒₘ), 6.75 (d, J=8.5 Hz, 1H, Hₐᵣₒₘ). ¹³C-NMR (125 MHz, C₆D₆): δ −5.24 (CH₃, TBDMS), 12.62 (CH₂CH₃), 18.41 (C, TBDMS), 23.93 (CH₂CH₃), 25.63 (CH₂), 26.09 (CH₃, TBDMS), 27.70 (CH₃, Boc), 30.42 (CH₂), 44.26 (CH₂), 62.69 (CH₂), 70.52 (CH₂), 83.24 (C, Boc), 111.22 (C, C═CH₂), 123.46 (CH, arom), 127.55 (CH, arom), 127.80 (CH₂, C═CH₂), 131.55 (C, arom), 133.72 (C, arom), 150.47 (C, arom), 153.10 (C, arom), 169.67 (C, C═O), 195.17 (C, C═O).

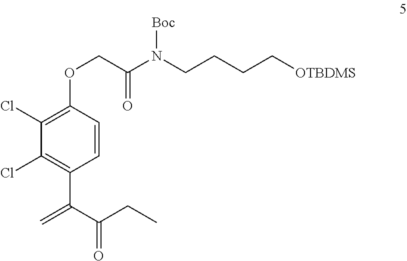 [SMILES: C=C(C(=O)CC)c1ccc(OCC(=O)N(CCCCO[Si](C)(C)C(C)(C)C)C(=O)OC(C)(C)C)c(Cl)c1Cl]

### 5. N-Boc-N-(4-hydroxybutyl)ethacrynic amide (compound of formula 4)

A solution of Tetra-n-Butylammonium Fluride (TBAF)/THF (1.16 mL, 1M, 2 eq), AcOH (0.066 mL, 1.16 mmol, 2 eq) in THF (10 mL) was added to a solution of the starting compound of formula 5 (340 mg, 0.58 mmol) in THF (10 mL). Stirring was allowed for 8 h. TLC (EtOAc/n-hexane 3/7) indicated the consumption of compound of formula 5 (R_{f}=0.77) and formation of the compound of formula 4 (R_{f}=0.27). The mixture was then concentrated at 40° C. under reduced pressure, followed by purification with flash chromatography using silica gel (50 g) with eluents of EtOAc/n-hexane=3/7 to provide a colorless oil compound of formula 4 in quantitative yield (270 mg). Anal. C₂₂H₂₉Cl₂NO₆, MW: 474.4, ESI+Q-TOF MS, M=473.1 (m/z), [2M+Na]⁺=970.9; the isotope clusters agree with the presence of Cl×2. HRMS-ESI, Calcd. C₂₂H₂₉Cl₂NO₆ [M]⁺: 473.13719. found: 473.13166. ¹H-NMR (500 MHz, C₆D₆): δ 0.99 (t, J=7.5 Hz, 3H, CH₂CH₃), 1.28 (s, 9H, H_{Boc}), 1.28. 1.32 (m, CH₂CH₂OH), 1.52 (q, J=7.5 Hz, J=7.0 Hz, (CON)CH₂CH₂), 2.43 (q, J=7.5 Hz, 2H, CCH₂CH₃), 3.25 (t, J=6.0 Hz, 2H, CH₂CH₂OH), 3.58 (t, J=7.0 Hz, 2H, (CON)CH₂CH₂), 5.01 (s, 2H, O(CH₂)CON), 5.26 (s, 1H, C═CH₂), 5.42 (s, 1H, C═CH₂), 6.27 (d, J=9.0 Hz, 1H, Hₐᵣₒₘ), 6.77 (d, J=9.0 Hz, 1H, Hₐᵣₒₘ). ¹³C-NMR (125 MHz, C₆D₆): δ 12.60 (CH₂CH₃), 23.90 (CH₂CH₃), 25.30 (CH₂), 27.67 (CH₃, Boc), 29.99 (CH₂), 44.18 (CH₂), 61.94 (CH₂), 70.50 (CH₂), 83.36 (C, Boc), 111.23 (CH, arom), 123.39 (C, C═CH₂), 126.97 (CH, arom), 127.80 (CH₂, C═CH₂), 131.53 (C, arom), 133.69 (C, arom), 150.45 (C, arom), 153.06 (C, arom), 156.45 (C, Boc), 169.84 (C, C═O), 195.33 (C, C═O).

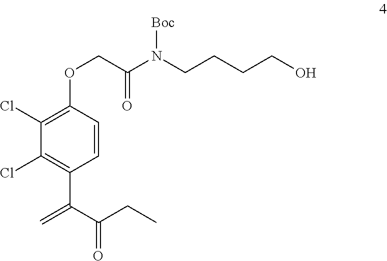 [SMILES: C=C(C(=O)CC)c1ccc(OCC(=O)N(CCCCO)C(=O)OC(C)(C)C)c(Cl)c1Cl]

### 6. N-Boc-N-[4-(toluenesulfonyloxy)butyl)ethacrynic amide (compound of formula 8)

In a preferred embodiment of the present invention, the compound of formula 2 can be the compound of formula 8. The detail process of the compound of formula 8 is described below. The starting compound of formula 4 (270 mg, 0.57 mmol) was dried through azeotropical distillation with toluene (1 mL×3) at 40° C. three times. After mixing with CH₂Cl₂ (10 mL), the mixture was moved to an ice bath and stirred for 5 min. A solution of Toluenesulfonyl (TsCl) (162 mg, 0.85 mmol, 1.5 eq) in CH₂Cl₂ (1 mL) and DMAP (139 mg, 1.13 mmol, 2 eq) were added sequentially. Stirring was allowed for 12 h. TLC (EtOAc/n-hexane=5/5) indicated the consumption of compound of formula 4 (R_{f}=0.45) and formation of compound of formula 8 (R_{f}=0.75). The mixture was then concentrated at 40° C. under reduced pressure followed by purification with flash chromatography using silica gel (50 g) with eluents of EtOAc/n-hexane 1/4 to provide colorless oil compound of formula 8 in 76% yield (271 mg). Anal. C₂₉H₃₅Cl₂NO₈S, MW: 628.6, ESI+Q-TOF MS, M=627.2 (m/z), [M+Na]⁺=650.4. HRMS-ESI, Calcd. C₂₉H₃₅Cl₂NO₈S [M]⁺: 627.14604. found: 627.14733. Anal. (C₂₉H₃₅Cl₂NO₈S) C, H, N; ¹H-NMR (500 MHz, C₆D₆): δ 0.98 (tt, J=7.5 Hz, 3H, CH₂CH₃), 1.23-1.25 (m, CH₂CH₂OTs), 1.28 (s, 9H, H_{Boc}), 1.38-1.44 (m, (CON)CH₂CH₂), 1.84 (s, 3H, CH₃, OTs), 2.43 (q, J=7.5 Hz, 2H, CCH₂CH₃), 3.44 (t, J=7.0 Hz, 2H, (CON)CH₂CH₂), 3.75 (dd, J=6.0 Hz, 2H, CH₂CH₂OTs), 4.99 (s, 2H, O(CH₂)CON), 5.27 (s, 1H, C═CH₂), 5.43 (s, 1H, C═CH₂), 6.27 (d, J=8.5 Hz, 1H, Hₐᵣₒₘ), 6.70 (d, J=8.5 Hz, 2H, CH, OTs), 6.79 (d, J=8 Hz, 1H, Hₐᵣₒₘ), 7.72 (d, J=8.5 Hz, 2H, CH, OTs). ¹³C-NMR (125 MHz, C₆D₆): δ 12.60 (CH₂CH₃), 21.09 (CH₃, OTs), 23.93 (CH₂CH₃), 24.79 (CH₂), 26.45 (CH₂), 27.70 (CH₃, Boc), 43.44 (CH₂), 69.66 (CH₂), 70.44 (CH₂), 83.67 (C, Boc), 111.19 (CH, arom), 123.47 (C, C═CH₂), 126.97 (CH, arom), 127.80 (CH₂, C═CH₂), 128.00 (CH, arom), 129.83 (CH, arom), 131.63 (C, arom), 133.85 (C, arom), 134.27 (C, arom), 144.31 (C, arom), 150.51 (C, arom), 152.87 (C, arom), 156.43 (C, Boc), 169.76 (C, C═O), 195.18 (C, C═O).

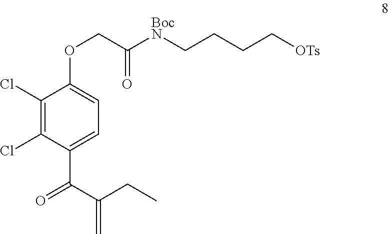 [SMILES: C=C(CC)C(=O)c1ccc(OCC(=O)N(CCCCOS(=O)(=O)c2ccc(C)cc2)C(=O)OC(C)(C)C)c(Cl)c1Cl]

### 7. N-Boc-N-(4-fluorobutyl)ethacrynic amide (compound of formula 9)

In a preferred embodiment of the present invention, the compound of formula 3 can be the compound of formula 9. The detail process of the compound of formula 9 is described below. The starting compound 4 (100 mg, 0.21 mmol) was dried through azeotropic distillation with toluene (1 mL) at 40° C. three times. After mixing with CH₂Cl₂ (5 mL), the mixture was stirred at −78° C. for 5 min. Diethylamino sulfur trifluoride (40 μL, 0.30 mmol, 1.5 eq) was then added and the mixture was stirred for 30 min. TLC (EtOAc/n-hexane 3/7) indicated the consumption of compound of formula 4 (R_{f}=0.40) and formation of the compound of formula 9 (R_{f}=0.60). After addition of satd. aqueous NaHCO₃ (10 mL), the organic layer was separated, and the aqueous layer was further extracted with CH₂Cl₂ twice. The organic layers were combined and dried over Na₂SO₄, followed by filtration through celite pad. The filtrate obtained was concentrated under reduced pressure at 40° C. The residue obtained was purified by flash chromatography using silica gel (30 g) with eluents of EtOAc/n-hexane 1/4 to provide colorless oil compound of formula 9 in 35% yield (35 mg). Anal. C₂₂H₂₈Cl₂FNO₅, MW: 476.4, ESI+Q-TOF MS, M=475.1 (m/z), [M+Na]⁺=498.0; the isotopic clusters agree with the presence of Cl×2. HRMS-ESI, Calcd. C₂₂H₂₈Cl₂FNO₅ [M]⁺: 475.13286. found: 475.13207. ¹H-NMR (500 MHz, C₆D₆): δ 0.92 (did, J=7.5 Hz, 3H, CH₂CH₃), 1.25 (s, 9H, H_{BOS}), 1.28 (dt, J=7.5 Hz, J=6.0 Hz, J_{H,F}=25.4 Hz, CH₂CH₂F), 1.48 (tt, J=7.5 Hz, J=7.0 Hz, (CON)CH₂CH₂), 2.43 (q, J=7.5 Hz, 2H, CCH₂CH₃), 3.51 (dd, J=6.0 Hz, J_{H,F}=48.0 Hz, 2H, CH₂CH₂F), 4.00 (t, J=7.0 Hz, 2H, (CON)CH₂CH₂), 4.99 (s, 2H, O(CH₂)CON), 5.25 (s, 1H, C═CH₂), 5.41 (s, 1H, C═CH₂), 6.24 (d, J=9.0 Hz, 1H, Hₐᵣₒₘ), 6.77 (d, J=9.0 Hz, 1H, Hₐᵣₒₘ). ¹³C-NMR (125 MHz, C₆D₆): δ 12.61 (CH₂CH₃), 23.92 (CH₂CH₃), 24.72 (J_{C,F}=3.8 Hz, CH₂CH₂CH₂F), 27.66 (CH₃, Boc), 27.90 (J_{C,F}=20.0 Hz, CH₂CH₂F), 43.75 (CH₂), 70.48 (CH₂), 82.41 (C, Boc), 83.42 (J_{C,F}=166.4 Hz, CH₂F), 111.21 (CH, arom), 123.49 (C, C═CH₂), 126.90 (CH, arom), 127.58 (CH₂, C═CH₂), 131.62 (C, arom), 133.85 (C, arom), 150.47 (C, arom), 152.92 (C, arom), 156.43 (C, Boc), 169.70 (C, C═O), 195.18 (C, C═O). ¹⁹F-NMR (470 MHz, C₆D₆): δ −218.23 (dd, J_{F,H}=25.4, J_{F,H}=48.0 Hz, 1F).

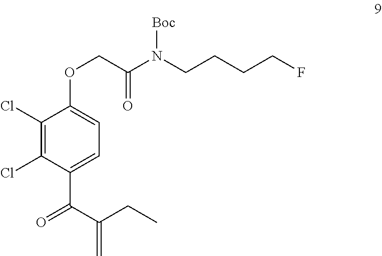 [SMILES: C=C(CC)C(=O)c1ccc(OCC(=O)N(CCCCF)C(=O)OC(C)(C)C)c(Cl)c1Cl]

### 8. N-(4-fluorobutyl)ethacrynic amide (FBuEA, compound of formula 10)

A solution of trifluoro acetic acid (250 μL) was added to a two-necked round-bottomed flask containing starting compound of formula 9 (30 mg, 0.063 mmol) in CH₂Cl₂ (2 mL). Stirring was allowed for 1 h. TLC (EtOAc/n-hexane 5/5) indicated the consumption of compound of formula 9 (R_{f}=0.70) and formation of compound (R_{f}=0.30). After addition of saturated aqueous NaHCO₃ (10 mL), the organic layer was collected and the aqueous layer was extracted with CH₂Cl₂ (2 mL×2). The organic layers combined were dried over Na₂SO₄ and filtered through celite pad. The filtrates were concentrated under reduced pressure, and the residue obtained was further purified by flash chromatography using silica gel (20 g) with eluents of EtOAc/n-hexane=5/5 to provide white solids formation of compound 10 in 70% yield (16 mg). Mp: 94-95° C. Anal. C₁₇H₂₀Cl₂FNO₃, MW: 376.3, ESI+Q-TOF MS, M=375.1 (m/z), [M+Na]⁺=398.0; the isotope clusters agree with the presence of Cl. HRMS-ESI, Calcd. C₁₇H₂₀Cl₂FNO₃ [M]⁺: 375.08043. found: 375.07974. Anal. (C₁₇H₂₀Cl₂FNO₃) C, H, N; ¹H-NMR (500 MHz, CD₃OD): δ 1.12 (t, J=7.5 Hz, 3H, CH₂CH₃), 1.65-1.73 (m, 4H, CH₂CH₂F and (CONH)CH₂CH₂), 2.44 (q, J=7.5 Hz, 2H, CCH₂CH₃), 3.33 (td, J=6.5 Hz, 2H, (CONH)CH₂CH₂), 4.37 (dt, J=5.5 Hz, J_{H,F}=48.9 Hz, 1H, CH₂CH₂F), 4.49 (dt, J=5.5 Hz, J_{H,F}=48.9 Hz, 1H, CH₂CH₂F s, 1H, C═CH₂), 4.69 (s, 2H, O(CH₂)CONH), 6.03 (s, 1H, C═CH₂), 6.59 (s, 1H, C═CH₂), 7.00 (d, J=8.5 Hz, 1H, Hₐᵣₒₘ), 7.24 (d, J=8.5 Hz, 1H, Hₐᵣₒₘ). ¹³C-NMR (125 MHz, C₆D₆): δ 12.62 (CH₂CH₃), 23.89 (CH₂CH₃), 25.81 (J_{C,F}=3.9 Hz, CH₂CH₂CH₂F), 27.79 (J_{C,F}=22.5 Hz, CH₂CH₂F), 38.53 (CH₂), 68.44 (CH₂), 83.05 (J_{C,F}=165.0 Hz, CH₂F), 111.12 (CH, arom), 122.82 (C, C═CH₂), 127.29 (CH, arom), 127.62 (CH₂, C═CH₂), 131.46 (C, arom), 134.50 (C, arom), 150.63 (C, arom), 154.65 (C, arom), 165.95 (C, C═O), 194.75 (C, C═O). ¹⁹F-NMR (470 MHz, C₆D₆): δ −217.82 (tt, J_{F,H}=25.9, J_{F,H}=48.9 Hz, 1F).

### 9. [¹⁸F]-N-(4-fluorobutyl)ethacrynic amide ([¹⁸F]FBuEA, compound of formula 1)

The radiolabeling of [¹⁸F]FBuEA was performed on a GEMS TracerLAB FX_{FN} synthesis module. On the GEMS TracerLAB FX_{FN} synthesis module, Hfluoride solution obtained from radiating H₂[¹⁸O]O (2 mL) in the warm room was loaded on a QMA-Light Sep-Pak cartridge (Waters), ¹⁸F ion obtained was eluted with Bu₄NHCO₃ (0.6 mL, 0.075M), and collected [¹⁸F]TBAF in the TracerLAB FX_{FN} glassy-carbon reactor. The mixture was distilled with CH₃CN (1 mL) for 2 mins. The residue was measured to be 8.6 GBq. A solution of compound of formula 8 (20 mg) in CH₃CN (1 mL) was added, and the mixture was heated to 120° C. for 10 min. A mixture was concentrated at 50° C. under reduced pressure and eluted with He gas for 2 min. Repeat the washing procedures once and obtain an intermediate compound of formula 9 with [¹⁸F]. A solution of TFA and CH₂Cl₂ (1 mL, v/v 1:5) was added to the mixture of compound of formula 9 with [¹⁸F], and stirring was allowed at 50° C. for 10 min. The solution was loaded onto a Al—N cartridge (Waters) setting comprising anionic exchange resin (DOWEX) and RC-18 plus (Waters), followed by eluting with acetone (8 mL). The filtrates (6 mL) were combined and purified with HPLC. HPLC settings: condition (A) ZORBAX SIL, 9.4×250 mm, 5 μm, EtOAc/n-hexane 1/2, Flowrate=3 mL/min, t_{R}=39.6 min (Radio); condition (B) CHEMCOSORB 7-ODS-H, 10×250 mm, 5 μm; eluent was set isocratically from CH₃CN/0.05% trifluoracetic acid=20/80 at 0 min to CH₃CN/0.05% trifluoracetic acid=95/5 at 10 min and a further gradient to CH₃CN (100%) at 20 min. Flowrate=3 mL/min, t_{R}=14.8 min (Radio). Fractions to [¹⁸F]FBuEA isolated from several injections were combined and concentrated to provide [¹⁸F]FBuEA (radiochemical yield of 44%, 3.8 GBq, decay corrected). Specific radioactivity and radiochemical purity were 48 GBq/μmole and 98%, respectively.

### 10. Preparation of Precursor of Compound of Formula 8

The preparation of the desired compound of formula 4 was initially started from a methyl ester of EA, which was prepared by using CH₂N₂ and EA (Scheme 1). Whereas the ester could be obtained in satisfactory yield 70%, subsequent coupling with the unprotected 4-aminobutyl alcohol provided the desired amide coupling product in only 20% yield due to the lack of regioselectivity and the less reactive ester. Hence, by adopting the usual HBTU-mediated amide coupling protocol in association with the source carboxylic acid and the well-protected O-TBDMS butyl amine compound 6, a satisfactory yield of 70% of amide compound 5 was obtained.

An attempt to protect the amide group with acetyl group using isoproprenyl acetate failed to provide the N-acetyl product and produced only the undesired O-acetyl byproduct, probably due to the instability of the silyl group (Scheme 2). Hence, through an alternative treatment with (Boc)₂O, the desired compound of formula 5 could be obtained in 76% yield. By removing the silyl group with the combination of tetrabutyl ammonium fluoride (TBAF) and AcOH, the desired product 7 could be obtained in quantitative yield. With the compound of formula 4 prepared, either the subsequent fluorination with DAST to provide the non-radioactive compound of formula 9 or the preparation of compound of formula 8 using TsCl can be performed. The cold compound of formula 9 and cold FBuEA were both used as authentic samples throughout the radiochemical synthesis for optimizing the radiochemical yield.

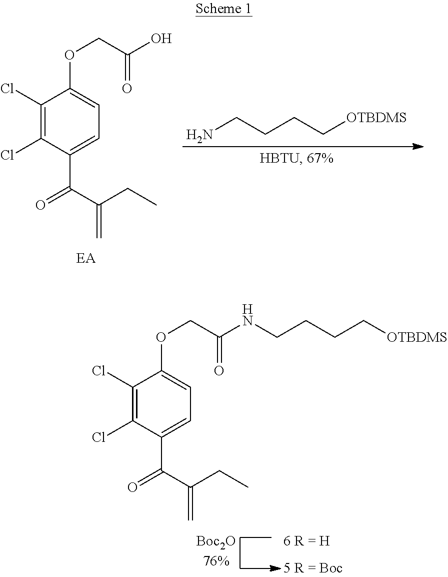 [SMILES: C=C(CC)C(=O)c1ccc(OCC(=O)O)c(Cl)c1Cl ; CC(C)(C)[Si](C)(C)OCCCCN ; C=C(CC)C(=O)c1ccc(OCC(=O)NCCCCO[Si](C)(C)C(C)(C)C)c(Cl)c1Cl]

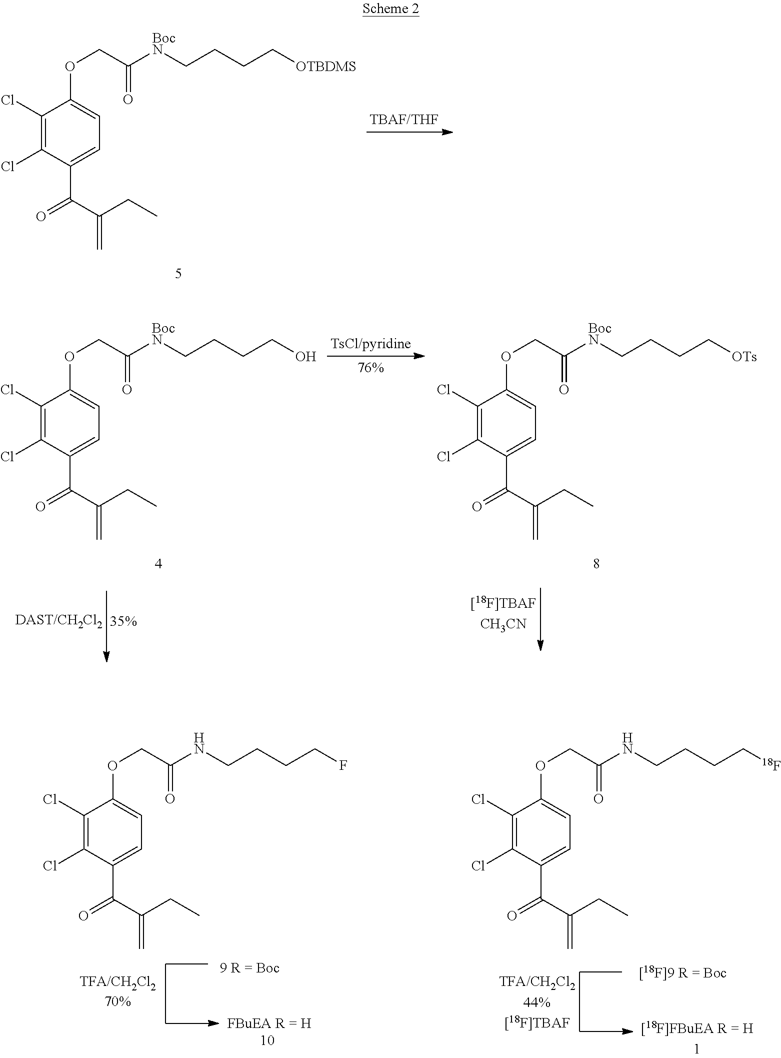 [SMILES: C=C(CC)C(=O)c1ccc(OCC(=O)N(CCCCO[Si](C)(C)C(C)(C)C)C(=O)OC(C)(C)C)c(Cl)c1Cl ; C=C(CC)C(=O)c1ccc(OCC(=O)N(CCCCO)C(=O)OC(C)(C)C)c(Cl)c1Cl ; C=C(CC)C(=O)c1ccc(OCC(=O)N(CCCCOS(=O)(=O)c2ccc(C)cc2)C(=O)OC(C)(C)C)c(Cl)c1Cl ; C=C(CC)C(=O)c1ccc(OCC(=O)NCCCCF)c(Cl)c1Cl ; C=C(CC)C(=O)c1ccc(OCC(=O)NCCCC[18F])c(Cl)c1Cl]

In order to obtain a satisfactory radiochemical yield from radiofluorination, it was critical to have sufficiently pure compound of formula 8. Therefore, samples were collected from centered fractionations of column chromatography with a number of compound of formula 8 preparations and the purity was met with criteria by elemental analysis.

### 11. Radiosynthesis of [¹⁸F]FBuEA (Compound of Formula 1)

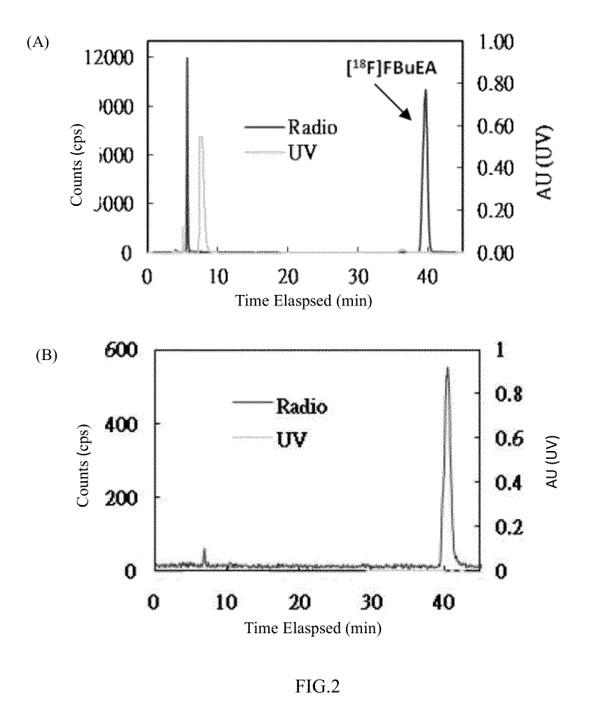

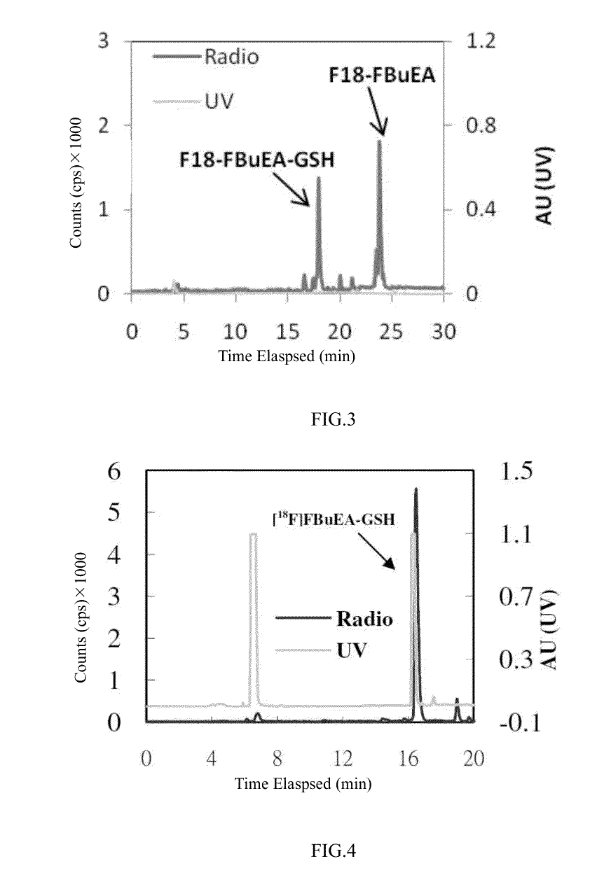

The preparation was carried out by using the tosylate compound 8 (20 mg) and [¹⁸F]F⁻N⁺Bu₄. Compound of formula 9 with [¹⁸F] was obtained in an average radiochemical yield of over 60%. The subsequent removal of the Boc group using trifluoro acetic acid (TFA) was accomplished. The HPLC chromatogram of the product mixture using a normal phase column showed an UV active peak at t_{R}=8.5 min, suggesting the released leaving group (FIG. 2(A)). Whereas this UV active substance and the nonpolar radioactive unknown substance (t_{R}=5.0 min) may not disturb the PET imaging outcomes of [¹⁸F]FBuEA, additional purification with semi-preparative HPLC was used, and the isolated fractions obtained reached a radiochemical purity of 98% and specific activity of greater than 48 GBq/μmol (FIG. 2(B)).

Interestingly, hydrolyzed byproduct or the byproduct from elimination was not observed in the chromatogram either before or after HPLC purification, which might be attributed to the previous manipulation of the cartridge settings. The present protocol for preparing [¹⁸F]FBuEA (ready for tail vein injection), which involved the two-step radiochemical synthesis including deprotection, collection of the fractions isolated from HPLC and concentration under reduced pressure was accomplished with a radiochemical yield of 44% (decay corrected) within 1.5 h (end of bombardment, EOB).

### Example 2

### Bioconjugating Experiment

### 1. Conjugation of Non-Radioactive FBuEA (Compound of Formula 10) and GSH at pH=8.0

The conjugation method was as previously described in the literature (Shi et al. (2006) J. Am. Chem. Soc. 128, 8459-8467). A solution of GSH (22 mg, 72 mmol, 1.5 eq) in distilled H₂O (1 mL) was added to a solution of compound of formula 10 (FBuEA)(18 mg, 48 μmol, 1 eq) in CH₃CN (1 mL). NaOH (50 mM, 1.5 mL) was added to adjust the pH value to 8. Stirring was allowed for 15 min. TLC indicated the consumption of the starting compound of formula 10 (R_{f}=0.9) and the formation of the product complex FBuEA-GSH(R_{f}=0.4). The mixture was filtered through Nylon (0.20 μM, National Scientific), and the resulting filtrate (3 mL) was purified using HPLC. The eluting condition was set at constant CH₃CN/0.05% trifluoracetic acid=20/80 for the first 1 min and then isocratically to a ratio of CH₃CN/0.05% trifluoracetic acid=40/60 at 11 min and a further gradient to CH₃CN (100%) at 20 min. Flowrate=3 mL/min, t_{R}=16.3 min (UV). The isolated fractions from a number of injections of HPLC were collected, followed by precipitation under the addition of CH₃CN (1 mL) to provide solids. The solid mixture was further filtered through gravity filtration followed by washing with cold CH₃CN. The residue thus obtained was dried under high vacuo at 40° C. to provide a white solid of FBuEA-GSH complex in 72% yield (21 mg). Cocrystallized solvents (e.g., H₂O or MeOH) were estimated to contribute a weight percent of 30% to 40%. Anal. C₂₇H₃₇Cl₂FN₄O₉S, MW: 682.2, LRMS, ESI+Q-TOF MS, M=682.2 (m/z), [M+H]⁺=683.2, [M+Na]⁺=705.1, [M+K]⁺=721.1; the isotopic clusters agree with the presence of Cl. Melting point: 127-128° C. HRMS-ESI, Calcd. C₂₇H₃₇Cl₂FN₄O₉S [M]⁺: 682.16423. found: 682.16389. ¹H-NMR (500 MHz, CD₃OD:D₂O=1:3, 50° C.): δ 0.87 (bs, 3H, CH₃), 1.65 (bs, 4H, CH₂CH₂), 1.71 (bs, 2H, CH₂), 2.14 (bs, 2H, CH₂), 2.52 (bs, 2H, CH₂), 2.75-3.04 (m, 4H, (CH₂SCH₂), 3.33 (bs, 2H, CH₂), 3.52 (bs, 1H, HCCO), 3.70 (bs, 1H, NCHCO), 3.74-3.82 (m, 2H, CH₂), 4.43 (bs, 1H, CH₂F), 4.74 (bs, 2H, OCH₂CO), 7.12-7.13 (m, 1H, Hₐᵣₒₘ), 7.59-7.62 (m, 1H, Hₐᵣₒₘ); ¹³C-NMR (125 MHz, CD₃OD:D₂O=1:3, 50° C.): 11.25 (CH₃), 25.18 (CH₂), 27.24 (CH₂), 28.00 (d, CH₂CH₂F, J_{C,F}=18.8 Hz), 32.59 (CH₂), 33.27 (CH₂), 34.90 (CH₂), 39.62 (CH₂), 44.31 (CH₂), 52.63 (CH), 54.14 (CH), 54.19 (CH), 55.22 (CH), 68.89 (CH₂), 85.63 (d, CH₂F, J_{C,F}=158 Hz), 112.57 (CH, arom), 124.17 (C, arom), 129.44 (CH, arom), 131.84 (C, arom), 134.07 (C, arom), 134.11 (C, arom), 156.84 (C, CO), 170.11 (C, CO), 172.31 (C, CO), 175.47 (C, CO), 175.51 (C, CO), 206.75 (C, CO). ¹⁹F-NMR (470 MHz, CD₃OD:D₂O=1:3, 50° C.): δ −218.16 (heptet, J_{F,H}=46.5, J_{F,H}=25.9 Hz, 1F).

### 2. Conjugation of [¹⁸F]FBuEA (Compound of Formula 1) and GSH at pH=8.0

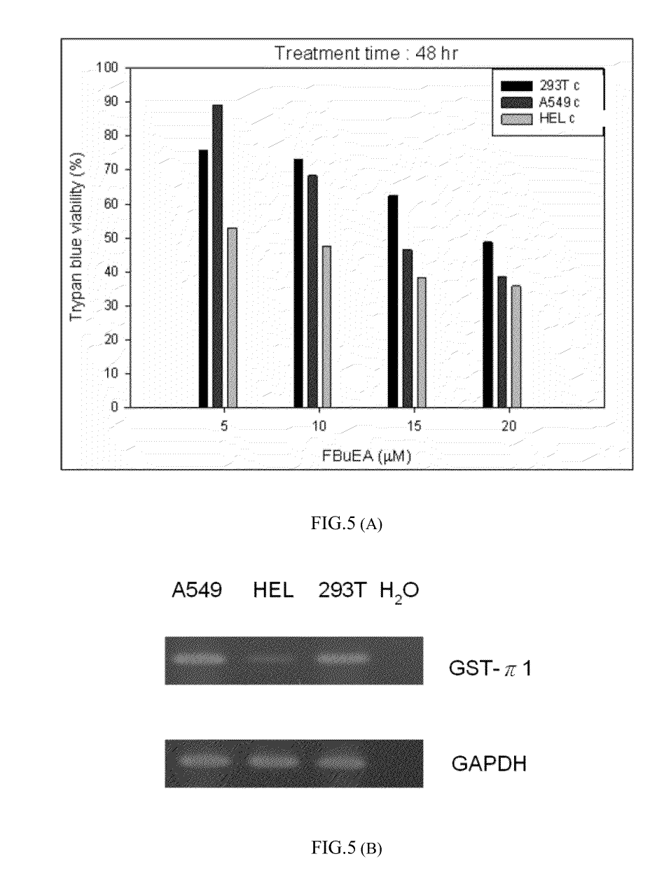

The conjugation method was according to the non-radioactive conjugation protocol as described above. HPLC-isolated [¹⁸F]FBuEA (1.1 MBq) in a round-bottom flask (25 mL) was concentrated under reduced pressure at 50° C. for 3 min. CH₃CN (1 mL) was added, and the azeotropic distillation was allowed for 5 min. CH₃CN (1 mL) and a solution of GSH (20 mg, 65 μmol) in distilled water (1 mL) were added sequentially. An aqueous solution of NaOH (50 mM, 0.6 mL) was added to adjust the pH to 8.0. Stirring was allowed for 15 min followed by HPLC analysis. A portion (0.4 mL) of the mixture (0.93 MBq, 3 mL), obtained from filtration through a 0.45 μM membrane filter, was drawn for HPLC injection. The eluting condition was the same as that described above for the non-radioactive preparation. The radiochemical yield of the product was 41% according to the calculation of the peak areas (FIG. 4). Specific activity was 10 GBq/μmol.

### 3. Conjugation of [¹⁸F]FBuEA (Compound of Formula 1) and GST Under Catalysis of GST-π

Enzymatic transformation of GSH to [¹⁸F]FBuEA was performed according to the protocol reported by Lo et al (Lo et al. (2007) Bioconjugate Chem. 18, 109-120). The fractions of [¹⁸F]FBuEA (8.1 MBq) isolated from HPLC purification was concentrated under reduced pressure at 50° C. for 20 min and the resultant residue was mixed with MeOH (0.1 mL). The following were added sequentially: an aliquot (0.1 mL) drawn from a solution of GSH (1 mg) in saline (1 mL), Na₃PO₄ buffer (1 mL, pH 7.0, 10 mM), and an aliquot (0.1 mL) drawn from a solution of GST-π protein (25 g) in Na₃PO₄ buffer (0.2 mL). The mixture was stirred at room temperature for 2 hr followed by addition of the quenching agent acetone (2 mL), and stirring was allowed for a further 2 min. After concentration under reduced pressure at 40° C. for 10 min, the mixture was washed with CH₂Cl₂ (2 mL) twice to collect the aqueous layer, and H₂O (1 mL) was used to extract the organic layer. The aqueous layers combined were submitted to HPLC analysis using eluting condition (B) as described above for preparation of both the non-radioactive and radioactive FBuEA-GSH complex. t_{R}=16.5 min (radio). The organic layer containing the most radioactivity (2.6 MBq) implied an incomplete consumption of the starting [¹⁸F]FBuEA. The complex [¹⁸F]FBuEA-GSH was obtained in a radiochemical yield of 16% (0.5 MBq, decay corrected).

### 4. The Effect of Cell Viability of [¹⁸F]FBuEA (Compound of Formula 1)

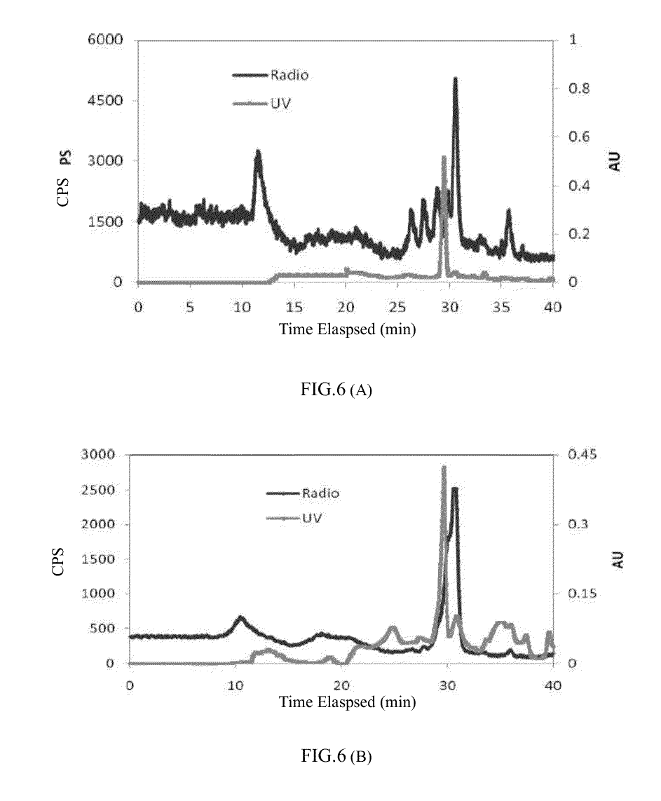

The protocol used was modified from that in the literature (George, L., Norman S. (1997). HPLC method for pharmaceutical analysis (Wiley-Interscience) (Fraga et al. (2011) Eur J. Med. Chem. 46, 349-355). Fractions of [¹⁸F]FBuEA (2 mL) isolated from reverse phase HPLC using condition (B) was added to the vial (10 mL). The mixture was concentrated under reduced pressure at 50° C. for 10 min. Toluene (1 mL) was added and distilled azeotropically twice. The cosolvent EtOH/saline (0.8 mL, 1:4 v/v) was added. The solution of [¹⁸F]FBuEA (6.3 MBq) was injected through the tail vein, and each of the blood samples (2 mL) was drawn from the femoral artery at 10, 30, 60 and 90 min. Following centrifugation at 3500 rpm for 5 min, the supernatant (0.5 mL) was mixed with CHCl₃ (2 mL) and H₂O (2 mL) under ultrasonic vibration for 5 min. The organic layer collected was eluted through a RC-18 cartridge (Waters) and washed with a cosolvent of MeOH/H₂O (3 mL, 1:4 v/v) to remove the undesired polar solutes, followed by eluting with CH₃CN (4 mL). The mixture obtained from each time point was then concentrated under reduced pressure at 40° C. for 10 min, and the residue obtained from each was mixed with a solution of authentic FBuEA in MeCN (200 μL drawn from 1 mg/2 mL) followed by filtration through a filter (Milipore, PTFE, 0.45 μm) for HPLC investigation. HPLC purification condition (B) as described above was adopted (FIG. 6(A) to (C)).

### 5. Octanol/Water Partition Coefficient

A lipophilicity test was carried out by measuring the log P value using P=C_{n-octanol}/C_{water} with the “Shake-flask method” (O. J. L. 383A) according to the official Journal of the European Community. n-octanol (2.5 mL) was added to a sample vial containing the isolated fraction of [¹⁸F]FBuEA (12.6 μCi) obtained from HPLC after concentration under reduced pressure. Stirring was allowed for 1 min, and an aqueous solution of PBS (0.01M, pH 7.3, 2.5 mL) was added. Vigorous stirring was continued for 15 min. The two layers were then separated, and three aliquots (0.5 mL×3) from each layer were drawn for counting in a gamma counter. The partition coefficient was log P=1.47±0.04

### 6. Analysis of Cytotoxicity

### Cell Line and Reagent

Human lung cancer cell line A549, human erythroleukemia cell line (HEL) and human embryonic kidney 293T cell line were cultivated in the RPMI-1640 medium (GIBCO) supplemented with 10% fetal bovine serum, 100 U/mL penicillin, 0.1 mg/mL streptomycin and 2 mM L-glutamine (GIBCO) at 37° C. in 5% CO₂ incubator. 293T was a non-tumorigenic cell line as a positive control in cell viability experiment.

### Cytotoxicity Study

To detect the cytotoxicity effects of FBuEA (compound of formula 10), A549 and HEL were treated with different concentration of FBuEA for 48 hr and compared with 293T. For the cytotoxicity study, trypan blue exclusion assay was performed to quantity the cell viability, 5×10⁴ cells and the medium with different concentration of FBuEA (from 0 to 20 M) were loaded in the 48-well plates for 48 hr. After 48 hr, cells mixed with trypan blue (GIBICO), just count the cells that have excluded the dye. Each count repeated three times and calculated the average of concentration. The cytotoxicity effects of EBuEA to A549, HEL and 293T cells were indicated by IC₅₀ value (comparing with untreated cells, the concentration of EBuEA required to decrease 50% cell viability).

Cytotoxic Effect Relative to mRNA Expression of GST-π1 (RT-PCR)

Total RNA of the cells was extracted by Easy Pure Total RNA Spin Kit (BIOMAN, INC.). To synthesize single-stranded cDNA from total RNA using High Capacity cDNA Reverse Transcription Kits (Applied Biosystems, INC) by protocol. To amplify cDNA using Thermo-start taq PCR MASTER MIX (THERMO, INC) in Thermal Cycler® PCR System 2720 (Applied Biosystems, INC) for 25 cycles, each cycle was include: denaturation 1 min at 95° C., annealing 1 min at 52° C. and extension 1 min for 72° C. The PCR primers are following: GAPDH-Forward, 5′-TGATGACATCAAGAAGGTGGTGAAG; GAPDH-Reverse, 5′-TCCTTGG-AGGCCATGTGGGCCAT; GST-π1-Forward, 5′-TCACTAAAGCCTCCTGC-CTAT-3′; GST-π1-Reverse, 5′-GCCTTCACATAGTCATCC-3′. Digital image of electrophoresis was performed by DigiGEL analysis systems. UN-SCAN-IT gel software (Silk Scientific) quantifies electrophoresis gel image. The ratio of GST-π1 to GAPDH was indicated the relative amount.

### 7. Preparation of Small Animal

### Cell Line

Lewis mouse lung carcinoma (LL2) cells were from Dr. Tsai-Yueh Luo at Institute of Nuclear Energy Research of Taiwan. The cells were cultivated in Dulbecco's modified Eagle's medium supplemented with 10% fetal bovine serum, 1% penicillin and streptomycin.

### Tumor Cells Inoculation

After trypsinization, LL2 cells were suspended in sodium phosphate (150 mM) and sodium chloride in phosphate buffer saline (PBS, pH 7.2) and stored on ice. The rats were anesthetized with intramuscular ketamine (60 mg/kg) and xylazine (8 mg/kg) and injected LL2 cells (2×10⁶) into its single region of the right leg with 30-gauge needle for 15 sec.

### 8. PET Image of Small Animal

The rats were anesthetized using 1 L/min 2% isoflurane (100% oxygen). After the rats had been anesthetized, they were given ¹⁸F-FBuEA (11 MBq) via the lateral tail vein. After the injection, the rats were fixed in the prone position on a carbon bed.

Dynamic imaging (0-120 min) single-frame scans were acquired with a small-animal PET camera (microPET R4; Concorde Microsystems Inc.). The region of interest (ROI) was disposed in the tumor region by hand, the image reconstructions of ROI were processed with ASIPro software (Concorde Microsystems INC.). Using microPET, the tumor volumes were determined by the intake of ¹⁸F-FBuEA (% ID/g).

### 9. Conjugation of Non-Radioactive FBuEA (Compound of Formula 10) and GSH at pH=8.0

The conjugation of FBuEA and GSH experiment was a straightforward, Michael addition reaction was very easy to accomplish. The FBuEA-GSH complex was obtained in a yield of 72%, and crystal water was accounted for 30%-40% yield. Thus, 50% of the FBuEA-GSH complex was a reasonable prediction.

### 11. Conjugation of [¹⁸F]FBuEA (Compound of Formula 1) and GSH at pH=8.0

The radiochemical yield (41%) of [¹⁸F]FBuEA-GSH complex from conjugation was less than the yield of the non-radiochemical control group (50%). This may be the crystallization solvents, such as H₂O, was not removed from FBuEA precipitate by CH₃CN as above-mentioned non-radioactive experiment, and the yield even less than that previously reported (Berndt et al. (2007)Nucl. Med. Biol. 34, 5-15, Wuest et al. (2003) Appl. Rad. Isot. 59, 43-48). In contrast to the radio TLC estimation reported by these literatures, the current yield calculation was based on the isolated product from HPLC purification. Therefore, as the non-radioactive experiment, the optimized yield may be 50%. The radioactivity of 10 GBq/μCi can carry out the animal imaging experiments. It was available a radioactivity of 1-10 GBq/μCi and several mCi of radioactivity by conjugation with other peptides and proteins.

### 12. Conjugation of [¹⁸F]FBuEA (Compound of Formula 1) and GSH Under Catalysis of GST-π

Enzymatic transformation of GSH to [¹⁸F]FBuEA was performed according to the protocol reported by Lo et al (Lo et al. (2007) Bioconjugate Chem. 18, 109-120). In the work, a prolonged reaction time (2 h) could assure that the reaction achieved completion. The conjugating experiment using [¹⁸F]FBuEA and GSH under catalysis by GST-π gave [¹⁸F]FBuEA-GSH 10 in 16% yield (FIG. 4). Under the reaction condition, such as reaction times, the concentration of substrates, may be modified by further experiments to optimize the radiochemical yield. As above-mentioned, it was most efficient at pH 8.0 due to the self-conjugation, and it was predictable that the efficiency reduces 10% at pH 7, therefore, 5% of yield is from self-conjugation and other 10% of yield was from conjugation with GST-π at pH 7.

### 13. The Cytotoxicity of [¹⁸F]FBuEA (Compound of Formula 1)

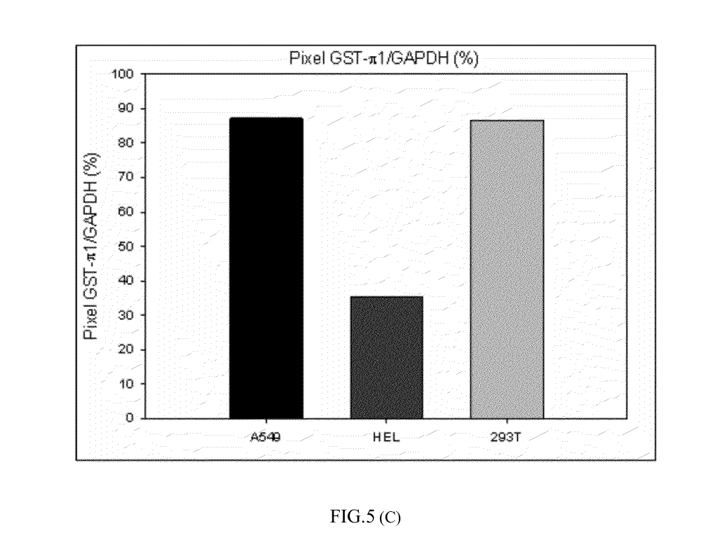

After treated with FBuEA, in contrast to A549 and 293T cells, HEL cells could be observed the inhibition of cell growth at a low concentration (IC₅₀: 5 μM) (FIG. 5(A)). A549 and 293T cells at a high concentration 14 μM and 20 μM respectively could be observed the relationship of cytotoxicity and dosage.

### 14. Cytotoxic Effect Relative to mRNA Expression of GST-π1

GST-π1 was a play an important role in detoxificaion of EA (Ethacrynic amide). Therefore, the present invention assumed that the cytotoxic effect was relative to GST-π1 expression, the mRNA expression of GST-π1 was determined by Half Quantity RT-PCR. The results showed the mRNA expression level of GST-π1 in HEL cells was lower than A549 and 293 cells (FIGS. 5(B) and 5(C)), they implied that EA analog was more toxic to the cells with less GST-π1. For cytotoxic effect, FBuEA has been found cytotoxic to the cancer cells, especially in the cells with low GST-π1 expression. Though EA-induced anti-tumor effects have been considered relative to (β-catenin cascade (Lu et al. (2009) PLoS One 4, e8294), GST-π1 could be play an important role in Cytotoxicity of EA analog.

### 15. The Effect of Cell Viability of [¹⁸F]FBuEA (Compound of Formula 1)

Before carrying out the animal imaging experiment, the stability of [¹⁸F]FBuEA in vivo was assessed by HPLC measurement of the radioactivity remaining in the blood. The in vivo half-life of [¹⁸F]FBuEA was determined to be 46 min (t_{1/2}). Compared to the plasma half-life of 0.5-1 h of the parent ethacrynic acid, the hydrophobic butyl moieties of [¹⁸F]FBuEA did not significantly alter the duration time (FIG. 6(D)).

### 16. PET Image of Small Animal

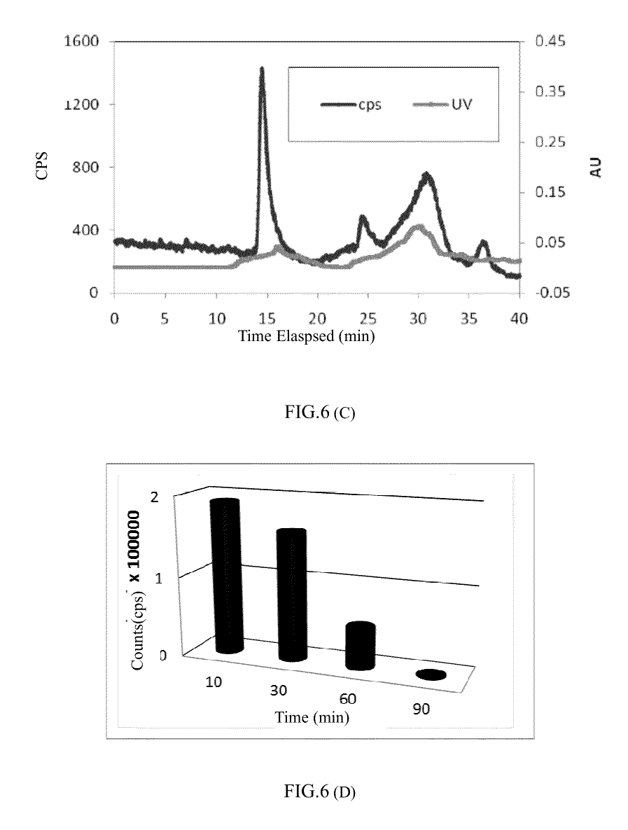

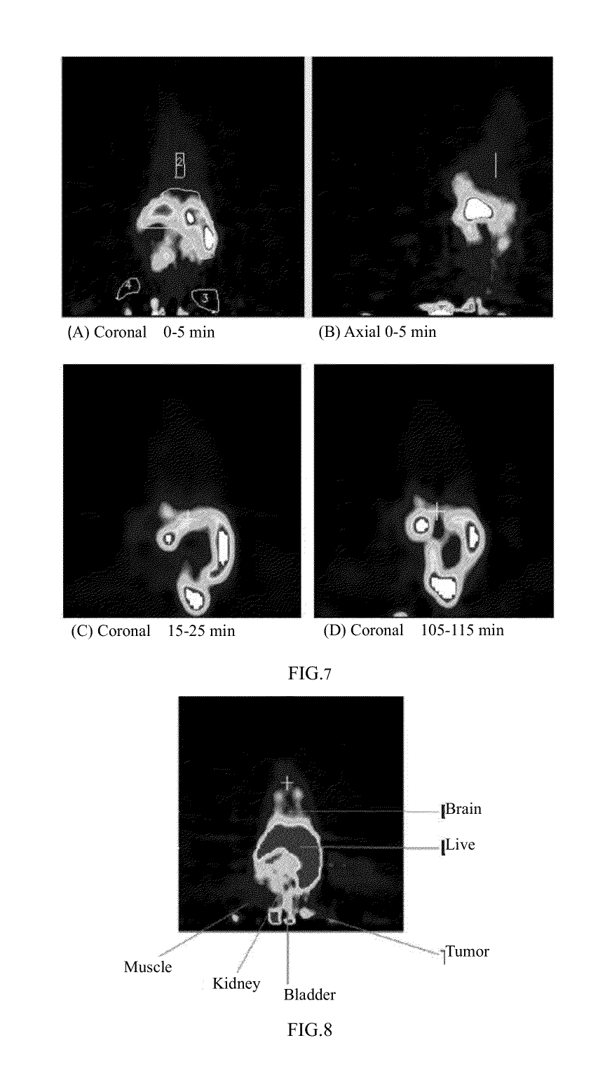

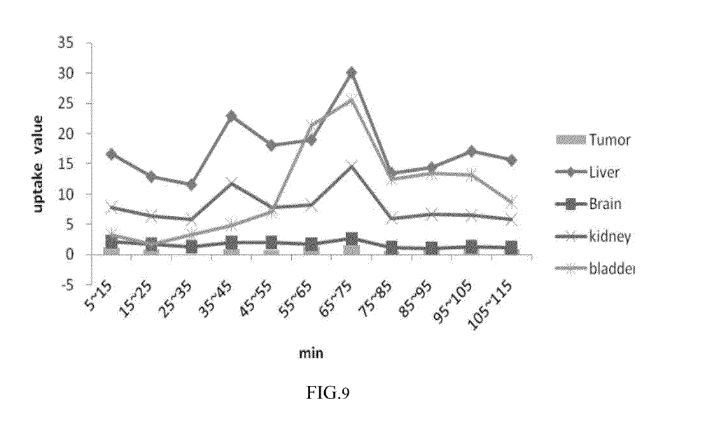

The biodistribution and dynamic images of LL2-induced small animal were assessed by PET image study. FIG. 7 showed small animal PET images averaged from 0 to 5 min, 15 to 25 min, 105 to 115 min timeframes postinjection of [¹⁸F]FBuEA. The images of time graph versus activity in the tumor, liver, brain, kidney and bladder were taken from the image date as the region of interest (ROI) (FIGS. 8 and 9). After intravenous application of [¹⁸F]FBuEA, the radiotracer was rapidly distributed. The liver was the main site of accumulation of [¹⁸F]FBuEA, which could be explained by the formation of the [¹⁸F]FBuEA-GSH complex as well as its subsequent transformation by membrane transporters. Although [¹⁸F]FBuEA was initially assumed to be capable of intracellular accumulation in tumors, a vast abundance of GSH and GST in liver could overwhelmingly dominate the formation of [¹⁸F]FBuEA-GSH complexes. In present dynamic images, especially in liver, it must clear the interaction of metabolism and membrane transporters between [¹⁸F]FBuEA and phase I, phase II in the further study.

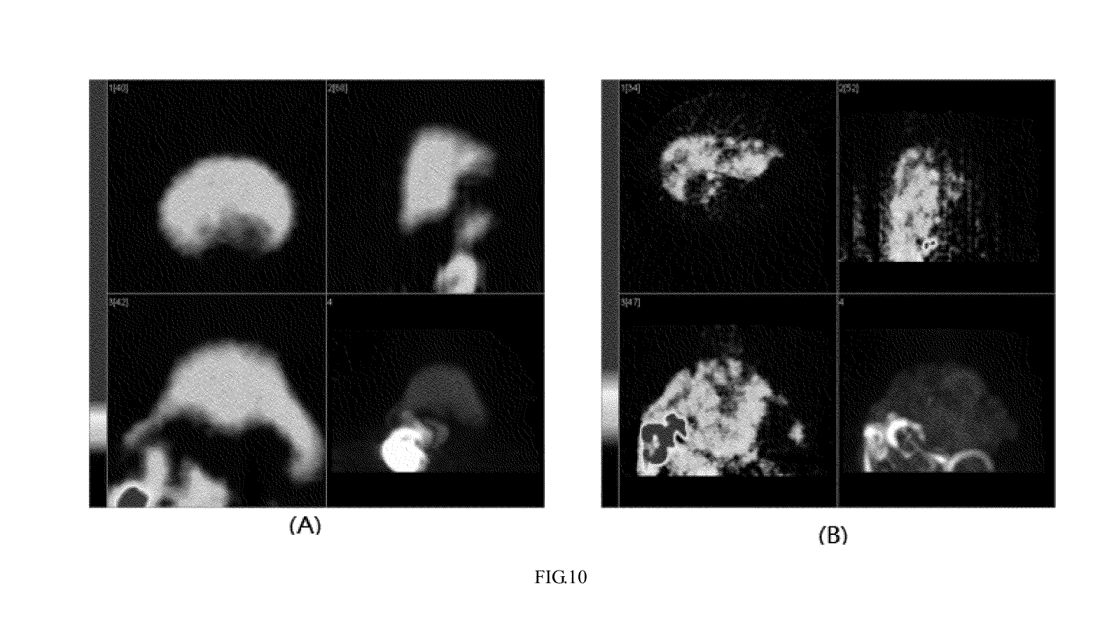

As shown in FIG. 10(A) and FIG. 10(B), the accumulation of [¹⁸F]FBuEA was as homogeneously distributed over the liver of the normal rat (FIG. 10(A)). By contrast, a heterogeneous distribution of [¹⁸F]FBuEA in the liver of the CCA rat was noted (FIG. 10(B)). The cold spots with no radioactivity accumulation suggested a low level of GST. The two rats were then sacrificed and their photographs showed significantly pathological differences. The initial assessment of [18F]FBuEA showed a negative correlation between GST isoenzymes and the tumor progression of the infiltrative liver of CCA rat.

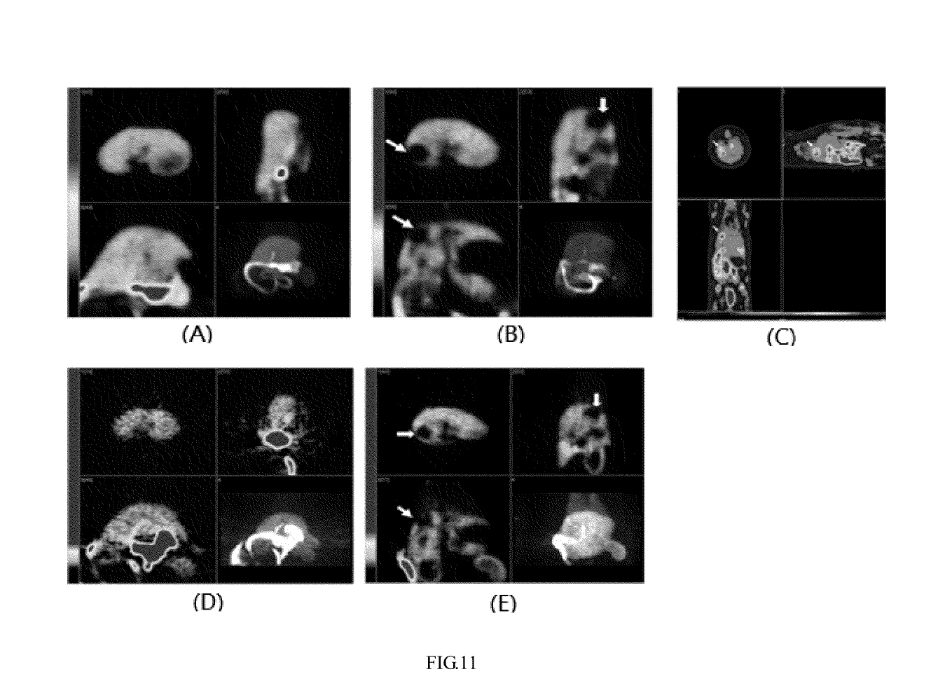

Being a promising imaging probe, [¹⁸F]FBuEA should be capable of detecting a disease at its early stage. Since an oversaturation of the imaging signal of normal rat was observed with a timeframe (FIG. 10(A)), a shorter imaging time frame was adopted. As shown in FIG. 11(C), PET images of CCA-rat receiving TAA (thioacetamide) for 18 weeks using [¹⁸F]fluorodeoxyglucose ([¹⁸F]FDG) indicated a significant hot spot implying a tumor lesion. Then the CCA rat along with the normal rat was subsequently imaged with [¹⁸F]FBuEA 5 days later. As shown in FIG. 11(B), the same region of the CCA rat that highlighted by [¹⁸F]FDG showed a cold spot instead and with a more diffused signal pattern that suggested either a deficiency in GST expression or an extraordinary function of GST. The same CCA rat after feeding for 23 weeks of TAA was imaged again with [¹⁸F]FBuEA but the imaging time was shortened to 5-10 min for optimization test (FIG. 11(D) and (E)). Whereas the tumor lesion was still dark and the cold spot was similar to that of 18 week rat. From the above comparisons, the optimized imaging sampling times appeared to be 0-30 min. These results suggested that regulation of GST-alpha synthesis was disturbed.

In brief, the liver is the major organ for the tracer uptake, and glutathione and GST enzymes play a role in the metabolism of this tracer. An in vivo half-life for [¹⁸F]FBuEA obtained from a preliminary in vivo stability test for [¹⁸F]FBuEA is shorter than the half-life of ¹⁸F. The adequate clearance rate is capable of providing an acceptable contrasting image for the TAA-treated CCA rat. An extraordinary change in the liver image was observed in the CCA rat at the early stage of tumor development and suggested its diagnostic potential. Therefore, [¹⁸F]FBuEA and [¹⁸F]FBuEA-GSH can applied in PET image for animal models in liver cancer research and disease models in liver disease (i.e., cirrhosis).

What is claimed is:

1. A method for preparing the compound of formula 1: 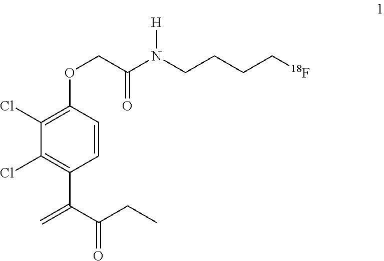 [SMILES: C=C(C(=O)CC)c1ccc(OCC(=O)NCCCC[18F])c(Cl)c1Cl]
comprising: (a) reacting the compound of formula 2 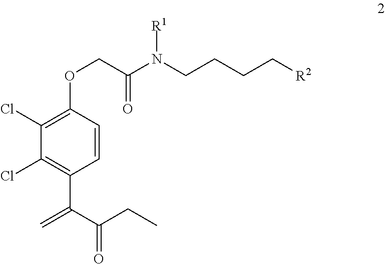
with a ¹⁸F-labeled fluorine reagent and acetonitrile to form the compound of formula 3; and 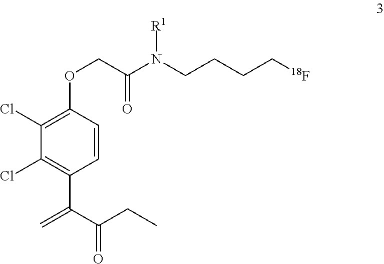
(b) using the compound of formula 3 with trifluoro acetic acid and haloalkanes to form the compound of formula 1, wherein R¹ is a protecting group of amide functional group and R² is a leaving group.

2. The method according to claim 1, wherein the protecting group of amide functional group is tert-butoxycarbonyl; and the leaving group is tosyloxy, methanesulfonyl, trifluoromethanesulfonyloxyl or bromine;

3. The method according to claim 1, wherein the ¹⁸F-labeled fluorine reagent is ¹⁸F-labeled tetrabutyl ammonium fluoride.

4. The method according to claim 1, wherein the compound of formula 2 is formed by reacting the compound of formula 4 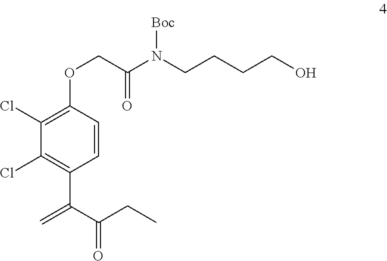 [SMILES: C=C(C(=O)CC)c1ccc(OCC(=O)N(CCCCO)C(=O)OC(C)(C)C)c(Cl)c1Cl]
with toluenesulfonyl chloride and a pyridine compound, wherein Boc is tert-butoxycarbonyl.

5. The method according to claim 4, wherein the pyridine compound is 4-(dimethylamino)pyridine.

6. The method according to claim 4, wherein the compound of formula 4 is formed by reacting the compound of formula 5 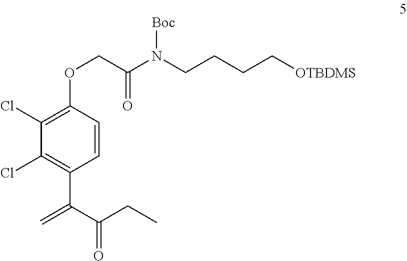 [SMILES: C=C(C(=O)CC)c1ccc(OCC(=O)N(CCCCO[Si](C)(C)C(C)(C)C)C(=O)OC(C)(C)C)c(Cl)c1Cl]
with tetrabutyl ammonium fluoride and acetic acid, wherein Boc is tert-butoxycarbonyl; OTBDMS is tert-butdimethoxysilane.

7. The method according to claim 6, wherein the compound of formula 5 is formed by reacting the compound of formula 6 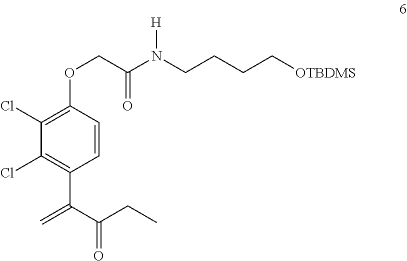 [SMILES: C=C(C(=O)CC)c1ccc(OCC(=O)NCCCCO[Si](C)(C)C(C)(C)C)c(Cl)c1Cl]
and di-tert-butyl dicarbonate, wherein OTBDMS is tert-butdimethoxysilane.

8. The method according to claim 7, wherein the compound of formula 6 is formed by reacting ethacrynic acid with N-Boc-N-[4-(t-butyldimethylsilanyloxy)-butyl-1-amine.

9. A composition for positron emission tomography (PET) imaging, comprising the compound of formula 1 according to claim 1 and a pharmaceutically acceptable carrier.

10. The composition according to claim 9, wherein the positron emission tomography (PET) imaging is used in an animal model of a liver tumor or a liver disease.

11. The composition according to claim 10, wherein the liver disease is cirrhosis.
